(19) United States
(12) Patent Application Publication
(10) Pub. No.: US 2012/0209621 A1
(43) Pub. Date: Aug. 16, 2012

(54) SKIN PHOTO INTERPRETING PROGRAM AND PROFESSIONAL & PHYSICIAN ADVISORY BOARD GUIDED DERMATOLOGY TEXT SEARCH ENGINE AND FACILITATOR OF PHYSICIAN APPOINTMENTS

(76) Inventors: Howard Green, Palm Beach Gardens, FL (US); Joseph Gene Cannata, JR., Cary, NC (US)

(21) Appl. No.: 13/206,448

(22) Filed: Aug. 9, 2011

Related U.S. Application Data
(60) Provisional application No. 61/401,245, filed on Aug. 10, 2010.
(60) Provisional application No. 61/519,446, filed on May 23, 2011.

(51) Int. Cl. A61B 5/00; G06Q 50/22

(52) U.S. Cl. 705/2; 600/300

(57) ABSTRACT

A method of providing information executed by a computer program interacting with several professionals and with a customer who has provided the photo of a person's skin, hair or nails, including the steps of: the customer acquiring at least one photo of a skin, hair or nails; the customer accessing the program on a computer; the customer subscribing to the text match service; the customer uploading to the program the photo; the customer entering an identification of the body area of which the photo was taken; the program publishing the photo and making it anonymous; the program queuing the photo for reading; a professional retrieving the photo from the queue; the professionals reading the photo and identifying the displayed skin condition; the professionals each matching the photo to two preferred stored texts describing the identified skin condition; the program publishing the photo in combination with the text; the program notifying the customer and the customer displaying the text. A preferred additional step is that of the program providing the customer with a random code to be used to expedite an appointment with a dermatologist in the customer's geographic area.

### FILING HISTORY

This application continues from provisional patent application Ser. No. 61/401,245, filed on Aug. 10, 2010, and from provisional patent application No. 61/519,446, filed on May 23, 2011.

### BACKGROUND OF THE INVENTION

1. Field of the Invention

The present invention relates generally to the field of dermatology searches. More specifically the present invention relates to a method of providing information executed by a computer program interacting with at least one and preferably several dermatology physicians and non-physician professionals referred to herein simply as professionals and with a customer who has provided a photograph (hereinafter photo) of his skin or her skin or another person's skin to interpret the photograph and any anomaly (if any exists) present on skin, hair or nails shown in the photo. (Skin, hair and nails represent the integument system, which covers all of dermatology searches.) The method includes the essential steps of: the customer acquiring at least one skin photo; the customer accessing the program on a computer; the customer subscribing to the text match service to gain further access to the program; the customer uploading to the program the photo; the customer entering an identification of the body area of which the photo was taken; the program publishing the photo; the program making the photo anonymous; the program queuing the photo for reading; several professionals retrieving the photo from the queue; the several professionals interpreting and identifying the photo; the professionals each matching the photo to stored texts identifying or describing the skin photo; the program determining the two leading text matches selected most frequently by the several professionals; the program publishing the photo in combination with the two leading matched texts such as one of: the Internet, email, cell phone text, postal mail and telefax; the program notifying the customer that the match is complete and the photo in combination with matched texts is available; and the customer displaying the photo and matched text. The customer receiving a random code and doctors names and contact information in order to facilitate and expedite an appointment with a dermatologist in the customers geographic area.

The method, including additional preferred steps, comprises the steps of: the customer acquiring at least one skin photo; the customer accessing the program on a computer; the program displaying a disclaimer; the program displaying a notice of privacy practices; the customer entering a decision of whether he or she accepts the disclaimer; if the customer rejects the disclaimer, the program rejecting the customer and ending his or her program use; if the customer accepts the disclaimer, a customer subscribing to the text match service to gain access to the program by entering a registration authorization and history in the form of a Protected Photographic and History Information (hereinafter referred to as PPHI) form and completing a customer profile including the customer address; the customer uploading the photo to the program; the customer entering an identification of the body area from which the photo was taken; the program displaying one of a tutorial and a how-to guide; the program publishing the photo; the customer entering credit card charging information to pay for use of the program; the program verifying whether in the event of expedited service, the credit card charging is authorized; the program making the photo anonymous; the program queuing the photo for reading; several professionals on the advisory board retrieving the photo from the queue; the several professionals determining whether the photo and history provided by the customer contains sufficient information to interpret; the several professionals interpreting the photo the professionals matching the photo two stored texts, preferably both first and second text choices, by most of the professionals matching or describing the skin photograph; the program publishing the photo in combination with the two matched texts most selected by the several professionals to a receiving device or through other medium of communication such as to one of the Internet, email, cell phone text, postal mail and telefax; the program notifying the customer that the match is complete and the matched texts are available; and the program displaying the photo in combination with the matched texts to the customer; the program providing a random anonymous code and contact information for the customer to use in order to expedite an appointment to see a dermatologist in the customer's geographic area; local dermatologists enrolled in the program receiving a matching random anonymous customer code via email, text, or fax to enable their office staff to recognize the customer and expedite an appointment for the customer. A still further step is the program billing a non-subscribing customer for use of the program. An optional further step is the program billing the customer for the matched texts if the matching is done on an expedited basis. Yet additional preferred steps are provided including: the program advertising local professionals in the geographical area of the customer, based on customer address provided as part of the customer profile (PPHI) information; the program displaying advertisements such as of drugs relating to the text matches; and the program referring the customer to a monitored community site.

A method of professional selection and interaction with the program is further provided, including the steps of the professional accessing the program on a computer; the professional becoming a member; the professional entering his or her qualifications for credentialing; evaluating the professional's qualifications including an on-line examination; if professional is rejected for performing text matching, the professional's interaction with the program ending; if professional is accepted to perform text matching, the programming creating a profile of the professional with account information; the accepted professional subscribing to the customer queue; all accepted professionals, referred to herein as the advisory board, subscribing to the customer photo queue; the accepted professionals accessing a photo from the queue; and the program statistically identifying the most selected first choice and the most selected second choice of matched texts.

Optional additional method steps include rating the accepted professionals on a daily, weekly, or monthly basis according to the frequency of participation in combination with frequency of concurrence with majority of professionals on the advisory board who participate in the particular photo text matches regarding the top two photo text matches; the program rewarding a certain number of the highest rated professionals on the advisory board selected according to the rating process.

2. Description of the Prior Art

The current standard for gathering information on a problem or question relating to skin from the Internet consists of two mechanisms involving lay people who (1) know the name of the skin finding and are searching for more information about what's on their or another person's skin, and people who (2) do not know what is on their or another person's skin who may be searching for information regarding what is on their or another person's skin these mechanisms being:

1. A layperson searching for textbook or atlas photos or medical information based on a diagnosed disease name. There are probably hundreds of websites already in use, which provide this information simply matching a disease name to text and photos. Examples of such self guided websites; web MD, derm atlas, etc., for future reference.

2. A person searching websites to match textbook or clinical photos with what they see on their or another person's skin in order to name or label what they see.

In either case, the exchange of information is severely limited by the lay person or customer's ability to recognize, describe and match what's on his or her skin with that of a website photo or disease description. In addition, the data bank of the website which the customer is searching may be insufficient or poorly designed and written to provide people with what they need to know about what is on their or another person's skin. Thus this prior art is the Internet version of the Merck Manual, which was the layperson guide to medicine in the 1970's and 1980's.

Often, people who know the name of what's on their or another person's skin search this site and download an overload or excess of information, and are therefore unable to relate their specific skin finding to the information discussed. These people end up with reams of information (which they often bring into the professional's office, which may or may not apply in a specific case.

Professional's can be annoyed at this behavior because professionals have to spend time explaining why certain information does not apply in a specific case, in addition to outlining and explaining what is correct for their patient. On the other hand, people who are simply trying to find out what is on their skin are at best amateur medical sleuths who also encounter this overload of either pertinent or incorrect information. They do not possess a clinical algorithm, which takes a dermatologist 8 to 9 years of formal medical training to acquire to accurately identify what is on the skin photograph of that or another person, thereby wasting time and energy. Board certified Dermatology Doctors may expedite, narrow and accurately focus the customer's search for information about the submitted skin or photo by accurately and concisely providing appropriate information regarding the submitted skin photo for a particular skin finding as represented in a photo taken or otherwise acquired by the customer.

Dermatology is an ideal field for this reference search application because the program, in combination with a professional, can identify skin images visually and all studies have demonstrated that the ability of a group of dermatology doctors to interpret skin findings via a submitted photograph or image, to be at least as accurate as a live viewing. By using the clinical algorithms developed by a group of dermatologists and associated professionals, each with 3-4 years of formal training, the present program delivers more accurate and concise information in a timely fashion to customers searching for information about what's on their or another person's skin. In addition, the program will provide the customer with a means of facilitating and expediting an appointment with a qualified doctor in their geographic area, which currently does not exist. The present program therefore provides a novel new service via the Internet for the Internet and global community.

It is thus an object of the present invention to provide a computer program which is information executed and which interacts with several participating professionals simultaneously and with a customer who has provided the photo of either him or herself or another person, and thus is the first professionally guided search engine for the skin on the Internet.

It is another object of the present invention to provide such a computer program in which the exchange of information is not in any way limited by the ability of the lay person or customer to recognize, describe and match his or her skin photo with that of a website photo or textbook description.

It is still another object of the present invention to provide such a computer program which encompasses most if not all published skin findings or anomalies.

It is yet another object of the present invention to provide such a computer program which provides a customer an avenue to facilitate and expedite an appointment with a dermatologist in his or her geographic area.

It is finally an object of the present invention to provide such a computer program, which is easy to use, and of maximum reliability.

### SUMMARY OF THE INVENTION

The present invention accomplishes the above-stated objectives, as well as others, as may be determined by a fair reading and interpretation of the entire specification.

A method of providing a customer with information about what is shown on a submitted photo or image with a computer program interacting with at least one and preferably several dermatology physicians and non-physician professional's referred to herein simply as professionals and with the customer who has provided the skin photo of either himself or herself or another person, including the steps of: the customer acquiring at least one photo of skin, hair or nails; the customer accessing the program on a computer; the customer subscribing to the text match service to gain further access to the program; the customer uploading to the program the photo; the customer entering an identification of the body area of which the photo was taken and answering several questions about the photo; the program publishing the photo; the program making the photo and customers answers anonymous; the program queuing the photo for reading; each of several professionals retrieving the photo from the queue; the several professionals reading the photo and identifying the skin findings shown in the photo; the several professionals each matching the photo to two stored texts identifying or describing the identified skin condition; the program determining the two leading text matches selected most frequently by the several professionals; the program publishing the photo in combination with the two leading matched texts such as one of: the Internet, email, cell phone text, postal mail and telefax; the program notifying the customer that the match is complete and the matched texts are available; and the customer after accepting a disclaimer, displaying the matched text. In addition, the customer will receive an anonymized random code to be used to facilitate and expedite an appointment with a dermatologist in his or her geographical area. Also preferably provided area flow or loop which does credentialing for professionals wishing to participate in this method, and internal quality control which has other professionals reading the image and a computer program which rates the accuracy or concurrence of the reading professionals. This same program will also anonymously analyze other variables of website use including but not limited to customer demographics and epidemiology. Still further optionally provided is receipt of continuing professional education credits by the participating professionals due to their interpretation of skin photos, for reading and performing two best text matches, and for the quality control review by anonymous other participating professionals. This same program will also permit enrolled professionals to anonymously enter monitored community social sites to interact either solely with registered co-professionals, or with the website customers.

A method of providing information searched for using a photo or image of the skin of a person with a computer program interacting with a professional and with a customer who has provided the photo of either him or herself or another person, including the steps: of the customer acquiring at least one photo of the skin; the customer accessing the program on a computer; the program displaying a disclaimer; the program displaying a notice of privacy practices; the customer entering a decision of whether he or she accepts the disclaimer; if the customer rejects the disclaimer, the program rejecting the customer and ending his or her program use; if the customer accepts the disclaimer; a customer subscribing to the text match service to gain access to the program by entering a registration authorization and history in the form of a PPHI form and completing a customer profile including the customer address; the customer uploading the photo to the program; the customer entering an identification of the body area from which the skin photo was taken; the program displaying one of a tutorial and a how-to guide; the program publishing the photo; in the event of expedited access the program will verify whether the credit card charging is authorized; the program making the photo anonymous; the program queuing the photo for reading; a professional retrieving the photo from the queue; the several professionals determining whether the photo and history provided by the customer contains sufficient information to interpret and identify a skin condition and identify what's on the skin in the photo; the professional looking at the photo and identifying what's on the skin in the photo; several professionals matching what's in the photo to textbook information; and choosing a majority of preferred participating professionals texts matches which may best describe what's on the photo; the program publishing the photo in combination with the top two matched texts to a receiving device or through other medium of communication such as to one of the Internet, email, cell phone text, postal mail and telefax; the program notifying the customer that the match is complete and the matched texts are available; the program displaying the matched texts to the customer; the program providing the customer with an anonymous random code and physician contact information to expedite an appointment with a dermatologist in the customer's geographic area. Optional additional method steps include the customer entering credit card charging information to pay for use of the program where the customer desires premium or expedited services. Alternatively to the method step of the customer entering credit card information, the program bills the customer for use of the expedited or premium services, till further optional additional step includes providing the customer with an expedited appointment with a dermatologist in the geographical area of the customer. The customer preferably is provided with an expedited appointment with the steps of the program providing the customer with contact information and a code with which to contact the dermatologist office; and if approved by the customer, providing the dermatologist office with a copy of the photo from the program and the contact code. No more personal information is transmitted in arranging the appointment.

### BRIEF DESCRIPTION OF THE DRAWINGS

Various other objects, advantages, and features of the invention will become apparent to those skilled in the art from the following discussion taken in conjunction with the following drawings, in which:

FIG. 1-4 are exemplary flow charts illustrating the present program.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### Method

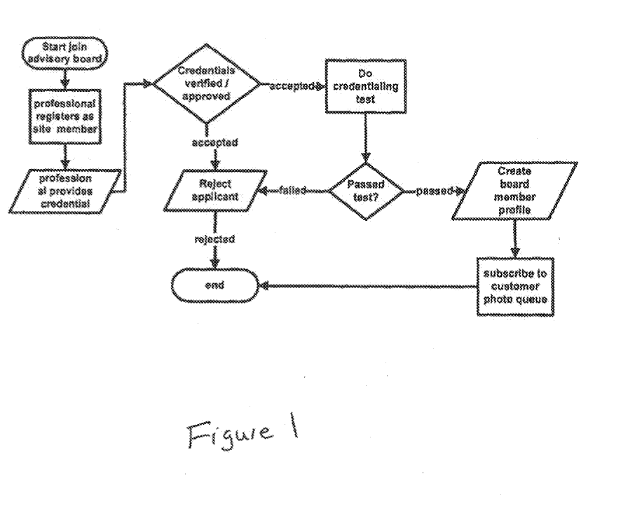

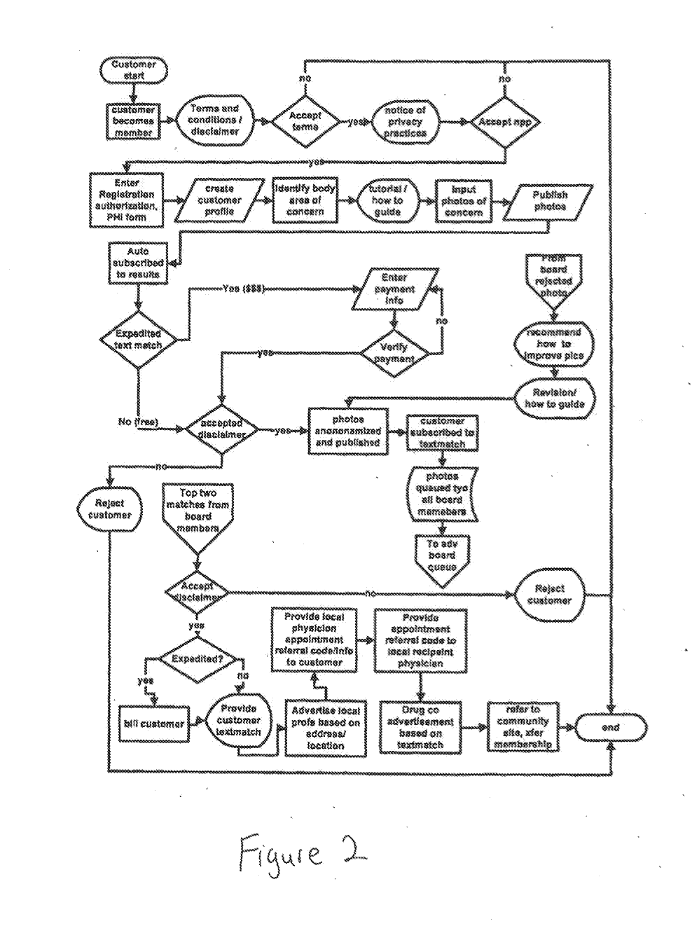

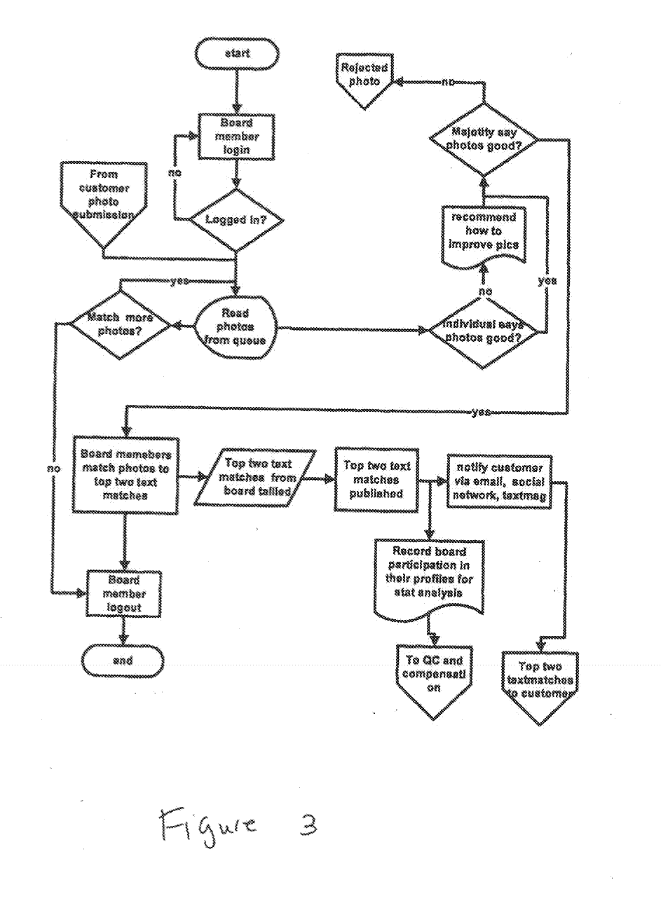

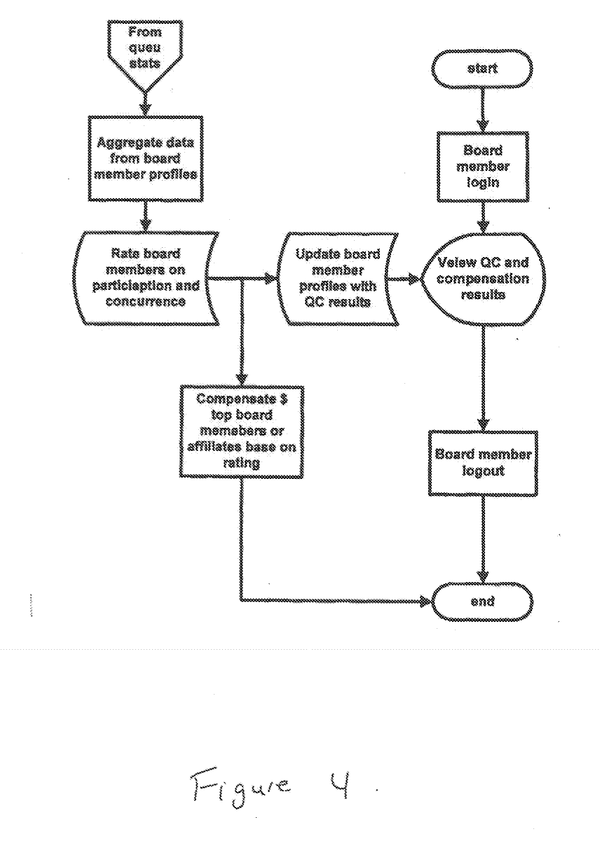

Referring to FIGS. 1-4, a method is disclosed of providing information executed by a computer program interacting with at least one and preferably several dermatology physicians and non-physician professionals all referred to herein simply as_professionals and with a customer who has provided the photo of skin, hair or nails (hereinafter photo) of either him or herself or another person to identify what's on the photo and any anomaly present on the skin shown in the photo. For this embodiment, customers are for the most part considered to be lay customers or members of the general public. The method includes the essential steps of: the customer acquiring at least one photo of the skin; the customer accessing the program on a computer; the customer subscribing to the text match service to gain access to the program; the customer uploading to the program the photo; the customer entering an identification of the body area of which the photo was taken; the program publishing the photo; the program making the photo anonymous; the program queuing the photo for reading; a professional providing credentials, a professional taking an online competency examination, the professional retrieving the photo from the queue; the several professionals reading the photo and identifying the skin finding in the photo; the several professionals each matching the photo to two preferred stored texts, preferably first and second preferred text match choices most likely correctly identifying what's on the skin shown in the submitted photo; the program determining the two leading text matches selected most frequently by the several professionals, preferably a first choice leading match and a second choice leading match; the program publishing the photo in combination with the two leading matched texts and an anonymous randomized code with local doctors' contact information such as one of: the Internet, email, cell phone text, postal mail and telefax; the program notifying the customer that the match is complete and the photo in combination with matched texts are available; and the customer displaying the photo and matched text.

The method, including additional preferred steps, comprises the steps of: the customer acquiring at least one skin photo; the customer accessing the program on a computer; the program displaying a disclaimer; the program displaying a notice of privacy practices; the customer entering a decision of whether he or she accepts the disclaimer; if the customer rejects the disclaimer, the program rejecting the customer and ending his or her program use; if the customer accepts the disclaimer; a customer subscribing to the text match service to gain access to the program by entering a registration authorization and historic information in the form of a PPHI form and completing a customer profile including the customer address; the customer uploading the photo to the program; the customer entering an identification of the body area from which the photo was taken; the program displaying one of a tutorial and a how-to guide; the program publishing the photo; the customer optionally entering credit card charging information to pay for use of the expedited or premium feature; the program verifying whether the credit card charging is authorized; the program making the photo anonymous; the program queuing the photo for reading; several professionals on the advisory board retrieving the photo from the queue; the several professionals determining whether the photo and history provided by the customer contains sufficient information to interpret and identify what's on the skin; the several professionals looking at the photo and determining what's on the skin shown in the photo; the professionals matching the condition identified from the photo to two stored texts, preferably first and second preferred matched text choices which match what's on the skin in the photo; the program publishing the photo in combination with the two leading matched texts most selected by the several professionals to a receiving device or through other medium of communication such as to one of the Internet, email, cell phone text, postal mail and telefax; the program notifying the customer that the match is complete and the matched texts are available; and the program displaying the photo in combination with the matched texts to the customer. still the program advertising local doctors or other health related services in the geographical area of the customer, based on customer address provided as part of the customer profile (PPHI) information; the program displaying advertisements such as of drugs relating to the text matches; and the program referring the customer to a monitored community site, refer for membership.

A method of professional selection and interaction with the program is further provided, including the steps of the professional accessing the program on a computer; the professional becoming a member; the professional entering his or her qualifications for evaluation; evaluating the professional qualifications; if professional is rejected for performing text matching, the professional interaction with the program ending; if professional is accepted to perform text_matching, the programming creating a profile of the professional with account information; the accepted professional subscribing to the customer queue; all accepted professionals, referred to herein as the advisory board, subscribing to the customer photo queue; the accepted professionals accessing a photo from the queue; and the program statistically identifying the most selected first choice and the most selected second choice of matched texts.

Optional additional method steps include rating the accepted professionals on a daily, weekly, or monthly basis according to the frequency of participation in combination with frequency of concurrence with majority of professionals on the advisory board who participate in the particular photo text matches regarding the top two photo text matches; the program rewarding a certain number of the highest rated professionals on the advisory board selected according to the rating process.

Still further optional additional step includes providing the customer with an expedited appointment with a dermatologist in the geographical area of the customer. The customer preferably is provided with an expedited appointment with the steps of the program providing the customer with contact information and an anonymous randomized code with which to contact the dermatologist office; and if the customer approves, providing the dermatologist office a copy of the photo from the program and the contact code. No more personal information is transmitted in arranging the appointment.

### Variation of Method

A variation of this method is provided, which is identical to the method described above, except that in place of customers as broadly defined above, only doctors will be provided access to the website/program to submit photos. To distinguish the doctors who are provided access to the program and submit photos for evaluation from the professionals on the advisory board or otherwise facilitating the operation of the program, an accessing doctor is referred to in the claims as a submitting doctor. It is further preferred that the submitting doctors be limited to only doctors who are invited by the website operators to access or join this doctors-only version of the website, such an invitation being a method step.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

I claim as my invention:

1. A method of providing information executed by a computer program interacting with several doctors and with a customer who has provided the photo of skin, hair or nails of either him or herself or another person, comprising the steps of:
the customer acquiring at least one photo of one of skin, hair and nails;
the customer accessing the program on a computer;
the customer subscribing to the text match service to gain access to the program;
the customer uploading to the program the photo;
the customer entering an identification of the body area of which the photo was taken;
the program publishing the photo;
the program making the photo anonymous;
the program queuing the photo for reading;
the several professionals each retrieving the photo from the queue;
the several doctors reading the photo and identifying the skin what if anything is on the skin, hair or nails shown in the photo;
the several professionals each matching the photo to two preferred stored texts identifying or describing the identified skin condition;
the program determining the two leading text matches selected most frequently by the several doctors;
the program publishing the photo in combination with the two leading matched texts most selected by the several professionals to a receiving device which is one of: the Internet, email, cell phone text, postal mail and telefax;
the program notifying the customer that the match is complete and the matched texts are available;
and the customer displaying the two matched texts.

2. A method of providing information executed by a computer program interacting with several doctors and with a customer who has provided the photo of either him or herself or another person, comprising the steps of:
the customer acquiring at least one photo of a skin hair, nails or mucosa;
the customer accessing the program on a computer;
the program displaying a disclaimer;
the program displaying a notice of privacy practices;
the customer entering a decision of whether he or she accepts the disclaimer;
if the customer rejects the disclaimer, the program rejecting the customer and ending his or her program use;
if the customer accepts the disclaimer;
a customer subscribing to the text match service to gain access to the program by entering a registration authorization, skin history and completing a customer profile including the customer address;
the customer uploading the photo to the program;
the customer entering an identification of the body area from which the photo was taken;
the program publishing the photo;
the customer entering credit card charging information to pay for use of the program;
the program verifying whether the credit card charging is authorized;
the program making the photo anonymous;
the program queuing the photo for reading;
a doctor retrieving the photo from the queue;
the several professionals determining whether the photo and history provided by the customer contains sufficient information to interpret and identify a skin condition;
the several professionals each reading the photo and identifying the skin condition shown in the photo;
the several professionals each matching the condition identified from the photo to two most preferred stored texts identifying or describing the condition;
the program determining the two leading text matches selected most frequently by the several professionals;
the program publishing the photo in combination with the two leading matched texts most selected by the several professionals to a receiving device which is one of: the Internet, email, cell phone text, postal mail and telefax;
the program notifying the customer that the match is complete and the matched texts are available;
and the program displaying the photo in combination with the matched texts to the customer.

3. The method of claim 2, comprising the additional step of:
the program advertising local doctors in the geographical area of the customer, based on customer address provided as part of the customer profile information.

4. The method of claim 3, comprising the additional step of: the program displaying advertisements.

5. The method of claim 4, wherein the advertisements are for one of: drugs, professionals, hospitals, clinics and non-prescribed products regardless of whether related to the text matches.

6. The method of claim 2, comprising the additional step of: the program displaying one of a tutorial and a how-to guide.

7. The method of claim 2, wherein the registration authorization and history is in the form of a PPHI form.

8. The method of claim 2, comprising the additional step of: the program referring the customer to a community site.

9. The method of claim 2, comprising the additional step of: the program for membership.

10. The method of claim 2, comprising the additional step of: a board certified doctor providing credentials.

11. The method of claim 2, comprising the additional step of: a doctor taking an online competency examination.

12. The method of claim 1, comprising the additional step of the program billing the customer for the matched texts if the matching is done on an expedited basis.

13. The method of claim 2, comprising the additional step of the program billing the customer for the matched texts if the matching is done on an expedited basis.

14. The method of claim 2, wherein the several doctors each select a first preferred matched text and a second preferred matched text.

15. Wherein the published two leading matched texts are categorized as a statistically first preferred matched text and a statistically second preferred matched text by the several professionals.

16. The method of claim 1, comprising the additional step of selecting professionals.

17. The method of claim 16, comprising the steps of professional selection and interaction with the program, comprising the steps of:
the professional accessing the program on a computer;
the professional becoming a member;
the professional entering his or her qualifications for evaluation;
evaluating the professional qualifications;
if professional is rejected for performing text matching, the professional interaction with the program ending;
if professional is accepted to perform text matching, the programming creating a profile of the professional with account information;
the accepted professional subscribing to the customer queue;
all accepted professionals subscribing to the customer photo queue;
the accepted professionals accessing a photo from the queue;
and the program statistically identifying the most selected first choice and the most selected second choice of matched texts by the professionals

18. The method of claim 17, wherein the professionals constitute an advisory board.

19. The method of claim 17, comprising the additional steps of:
rating the accepted professionals on a periodic basis according to the frequency of participation in combination with frequency of concurrence with majority of professionals on the advisory board who participate in the particular photo text matches regarding the top two photo text matches;
the program rewarding a certain number of the highest rated professionals on the advisory board selected according to the rating process.

20. The method of claim 19, wherein the periodic basis on which professionals are rated is one of: daily, weekly, and monthly.

21. A method of providing information executed by a computer program interacting with a professional and with a customer who has provided the photo of skin, hair or nails of either him or herself or another person, comprising the steps of:
the customer acquiring at least one photo of one of skin, hair and nails;
the customer accessing the program on a computer;
the customer subscribing to the text match service to gain access to the program;
the customer uploading to the program the photo;
the customer entering an identification of the body area of which the photo was taken;
the program publishing the photo;
the program making the photo anonymous;
the program queuing the photo for reading;
a professional retrieving the photo from the que;
the professional reading the photo and identifying the skin what if anything is on the skin, hair or nails shown in the photo;
the professional matching the photo to a stored text identifying or describing the identified skin condition;
the program publishing the photo in combination with the matched text such as one of: the Internet, email, cell phone text, postal mail and telefax;
the program notifying the customer that the match is complete and the matched text is available;
and the customer displaying the matched text.

22. A method of providing information executed by a computer program interacting with a professional and with a customer who has provided the photo of either him or herself or another person, comprising the steps of:
the customer acquiring at least one photo of a skin hair, nails or mucosa;
the customer accessing the program on a computer;
the program displaying a disclaimer;
the program displaying a notice of privacy practices;
the customer entering a decision of whether he or she accepts the disclaimer;
if the customer rejects the disclaimer, the program rejecting the customer and ending his or her program use;
if the customer accepts the disclaimer;
a customer subscribing to the text match service to gain access to the program by entering a registration authorization, skin history and completing a customer profile including the customer address;
the customer uploading the photo to the program;
the customer entering an identification of the body area from which the photo was taken;
the program publishing the photo;
the customer entering credit card charging information to pay for use of the program;
the program verifying whether the credit card charging is authorized;
the program making the photo anonymous;
the program queuing the photo for reading;
a professional retrieving the photo from the que;
the professional determining whether the photo and history provided by the customer contains sufficient information to interpret and identify a skin condition;
the professional reading the photo and identifying the skin condition shown in the photo;
the professional matching the condition identified from the photo to a stored text identifying or describing the condition;
the program publishing the photo in combination with the matched text to a receiving device or through other medium of communication such as to one of the Internet, email, cell phone text, postal mail and telefax;
the program notifying the customer that the match is complete and the matched text is available;
the program billing the customer for use of the program;
and the program displaying the photo in combination with the matched text to the customer.

23. A method of providing information executed by a computer program interacting with several professionals and with a photo submitting doctor who has provided the photo of skin, hair or nails of either him or herself or another person, comprising the steps of:
the submitting doctor acquiring at least one photo of one of skin, hair and nails;
the submitting doctor accessing the program on a computer;
the submitting doctor subscribing to the text match service to gain access to the program;
the submitting doctor uploading to the program the photo;
the submitting doctor entering an identification of the body area of which the photo was taken;
the program publishing the photo;
the program making the photo anonymous;
the program queuing the photo for reading;
the several professionals each retrieving the photo from the queue;
the several professionals reading the photo and identifying the skin what if anything is on the skin, hair or nails shown in the photo;
the several professionals each matching the photo to two preferred stored texts identifying or describing the identified skin condition;
the program determining the two leading text matches selected most frequently by the several professionals;
the program publishing the photo in combination with the two leading matched texts most selected by the several professionals to a receiving device which is one of: the Internet, email, cell phone text, postal mail and telefax;
the program notifying the submitting doctor that the match is complete and the matched texts are available;
and the submitting doctor displaying the two matched texts.

24. A method of providing information executed by a computer program interacting with several professionals and with a photo submitting doctor who has provided the photo of either him or herself or another person, comprising the steps of:
the submitting doctor acquiring at least one photo of a skin hair, nails or mucosa;
the submitting doctor accessing the program on a computer;
the program displaying a disclaimer;
the program displaying a notice of privacy practices;
the submitting doctor entering a decision of whether he or she accepts the disclaimer;
if the submitting doctor rejects the disclaimer, the program rejecting the submitting doctor and ending his or her program use;
if the submitting doctor accepts the disclaimer;
a submitting doctor subscribing to the text match service to gain access to the program by entering a registration authorization, skin history and completing a submitting doctor profile including the submitting doctor address;
the submitting doctor uploading the photo to the program;
the submitting doctor entering an identification of the body area from which the photo was taken;
the program publishing the photo;
the submitting doctor entering credit card charging information to pay for use of the program;
the program verifying whether the credit card charging is authorized;
the program making the photo anonymous;
the program queuing the photo for reading;
a submitting doctor retrieving the photo from the queue;
the several professionals determining whether the photo and history provided by the submitting doctor contains sufficient information to interpret and identify a skin condition;
the several professionals each reading the photo and identifying the skin condition shown in the photo;
the several professionals each matching the condition identified from the photo to two most preferred stored texts identifying or describing the condition;
the program determining the two leading text matches selected most frequently by the several professionals;
the program publishing the photo in combination with the two leading matched texts most selected by the several professionals to a receiving device which is one of: the Internet, email, cell phone text, postal mail and telefax;
the program notifying the submitting doctor that the match is complete and the matched texts are available;
and the program displaying the photo in combination with the matched texts to the submitting doctor.

25. A method of claim 23, comprising the additional step of: inviting the submitting doctor to access the computer program, while denying uninvited doctors access to the computer program.

26. A method of claim 24, comprising the additional step of: inviting the submitting doctor to access the computer program, while denying uninvited doctors access to the computer program.
